(19) United States
(12) Patent Application Publication
(10) Pub. No.: US 2022/0323224 A1
(43) Pub. Date: Oct. 13, 2022

(54) PATIENT-SPECIFIC ARTIFICIAL TEMPOROMANDIBULAR JOINT

(71) Applicant: MEDYSSEY CO., LTD., Chungcheongbuk-do (KR)

(72) Inventors: Ui Lyong LEE, Seoul (KR); Su Heon WOO, Gyeonggi-do (KR); Hyo Bok JEONG, Chungcheongbuk-do (KR); Hye Ji PARK, Seoul (KR); Hyun-Woo JUNG, Chungcheongbuk-do (KR); Dae Hyun SONG, Seoul (KR)

(73) Assignee: MEDYSSEY CO., LTD., Chungcheongbuk-do (KR)

(21) Appl. No.: 17/494,903

(22) Filed: Oct. 6, 2021

(30) Foreign Application Priority Data
Apr. 9, 2021 (KR) 1020210046417

(51) Int. Cl. A61F 2/30; A61B 17/80

(52) U.S. Cl. CPC A61F 2/3099; A61B 17/8052; A61B 17/8071; A61F 2/3094; A61F 2002/30991; A61F 2002/30993; A61F 2002/30383; A61F 2002/30578; A61F 2002/30985

(57) ABSTRACT

A patient-specific artificial temporomandibular joint is proposed. The patient-specific artificial temporomandibular joint includes: a temporal bone coupling part including a fixing body that is fixed to the temporal bone of a patient and a head receiver that is detachably coupled to the fixing body and provides a close-contact curved surface being open downward; and a mandible coupling part including a coupling body that is fixed to the mandible of a patient and a head that has a spherical shape and is supported by the coupling body in contact with the close-contact curved surface of the head receiver. The patient-specific artificial temporomandibular joint is manufactured by 3-D printing on the basis of shape data of the temporal bone or the mandible of a patient, so a good implanting effect is provided and the patient quickly recovers. Further, worn parts can be partially replaced, thus there is a little burden of maintenance.

### CROSS REFERENCE TO RELATED APPLICATION

The present application claims priority to Korean Patent Application No. 10-2021-0046417, filed Apr. 9, 2021, the entire contents of which is incorporated herein for all purposes by this reference.

### BACKGROUND OF THE INVENTION

The present disclosure relates to an artificial temporomandibular joint and, more particularly, a patient-specific artificial temporomandibular joint of which worn parts can be partially replaced and that is manufactured by 3-D printing on the basis of the shape data of the temporal bone and the mandible of a patient.

### DESCRIPTION OF THE RELATED ART

The temporomandibular joint, which supports the mandible when a mouth is opened or closed, is composed of the joint of the temporal bone, the bone head of the mandible, and a joint disc. The joint disc is positioned between a bone head and a joint, absorbs shock, and is connected to the surrounding ligaments. When the temporomandibular joint is normally operated, it is possible to yawn with the mouth open wide, masticate food, etc.

When the temporomandibular joint is not normally operated, a noise is made when the mouth is opened or closed, it is difficult to open the mouth, and a pain or inconvenience is generated when masticating food. As reasons of such temporomandibular disorder, there are malocclusion due to wrong habits, physical shock, etc., and as methods for treating temporomandibular disorder, there is physical treatment or medicinal treatment, or simply a method of holding a correcting device in the mouth.

However, when temporomandibular disorder is severe, a surgical operation is performed in some cases. That is, there is a surgical operation of fixing an artificial temporomandibular joint replacing the temporomandibular joint to the temporal bone and the mandible. The artificial temporomandibular joint replacement is a method that is applied when a treatment effect cannot be expected through other treatments, and as the related art, a customized artificial jaw joint unit has been disclosed in Korean Patent Application Publication No. 10-2020-0082700.

The disclosed artificial jaw joint includes: a first plate that is attached along the lower line of the mandible constituting the jaw joint and has an insertion groove open outward; a second plate that is attached to the temporal bone forming the jaw joint together with the mandible; and a main implant that is forcibly inserted when it approaches the first plate to be detachable from the first plate even without a specific fastener and that is disposed in a customized type on a surface facing the second plate.

### SUMMARY OF THE INVENTION

The present disclosure has been made in an effort to solve the problems and an objective of the present disclosure is to provide a patient-specific artificial temporomandibular joint that provides a good implanting efficiency because it is manufactured by 3-D printing and of which worn parts can be partially replaced.

In order to achieve the objectives, the patient-specific artificial temporomandibular joint of the present disclosure includes: a temporal bone coupling part including a fixing body that is fixed to the temporal bone of a patient and a head receiver that is detachably coupled to the fixing body and provides a close-contact curved surface being open downward; and a mandible coupling part including a coupling body that is fixed to the mandible of a patient and a head that has a spherical shape and is supported by the coupling body in contact with the close-contact curved surface of the head receiver.

The fixing body may be manufactured by a 3D printer or may be shaped through machining and may have a fixing plate that is brought in contact with the temporal bone and has several screw holes, and a holding supporter that is integrated with the fixing plate and has a coupling space on a bottom thereof; and the head receiver may be made of medical synthetic resin, has an insertion-fixing portion on a top thereof that is detachably mounted in the coupling space, and may have the close-contact curved surface on a bottom thereof.

The coupling body may be manufactured by 3D printing or may be shaped through machining and may have a fixing plate that is fixed to the mandible of a patient and has several screw holes, and an extension that is integrated with the fixing plate and extends under the head receiver.

The head may be made of medical synthetic resin and may be detachably coupled to an end of the extension.

A head support and a fixing protrusion that provide supporting force may be formed at the end of the extension, and a locking groove receiving the fixing protrusion to maintain coupling to the fixing protrusion may be formed at the head.

The coupling space may be a groove being open downward to receive the insertion-fixing portion, guide slopes that guide the insertion-fixing portion into the coupling space when the insertion-fixing portion is forcibly fitted into the coupling space may be formed around the coupling space, and supporting steps that prevent separation of the insertion-fixing portion fitted in the coupling space by supporting the insertion-fixing portion may be formed inside the coupling space.

Elastic locking steps, which are elastically deformed by reaction force applied from the guide slopes when the insertion-fixing portion is forcibly fitted into the coupling space and which are locked to the supporting steps when the insertion-fixing portion is fully inserted in the coupling space, may be formed at the insertion-fixing portion.

The coupling space may be a female thread hole being open downward and having a predetermined inner diameter and the insertion-fixing portion may be a male thread portion that is thread-fastened to the female thread groove.

In order to achieve the objectives of the present disclosure, a patient-specific artificial temporomandibular joint includes: a temporal bone coupling part including a fixing body that is fixed to the temporal bone of a patient and a head that has a spherical shape and is detachably coupled to a bottom of the fixing body; and a mandible coupler including a coupling body that is coupled to the mandible of a patient and a head receiver that is fixed to the coupling body and provides a close-contact curved surface that is in contact with the head.

The fixing body may be manufactured by a 3D printer or may be shaped through machining and may have a fixing plate that is brought in contact with the temporal bone of a patient and has several screw holes, and a holding supporter that is integrated with the fixing plate and provides a coupling space on a bottom thereof; and the head may be made of medical synthetic resin and may have an insertion-fixing portion on a top thereof that is detachably coupled to the coupling space.

The coupling body may be manufactured by 3D printing or may be shaped through machining and may have a fixing plate that is fixed to the mandible of a patient and has several screw holes, and an extension that is integrated with the fixing plate and extends under the head.

An adaptor having a coupling space being open upward may be formed at an extending end of the extension, and the head receiver may be made of synthetic resin and may have an insertion-fixing portion that is detachably coupled to the coupling space.

The coupling space may be a groove being open to be able to receive the insertion-fixing portion, guide slopes that guide the insertion-fixing portion into the coupling space may be formed around the coupling space, supporting steps that support the insertion-fixing portion fitted in the coupling space and prevent separation of the insertion-fixing portion may be formed inside the coupling space, and elastic locking steps, which are elastically deformed by reaction force applied from the guide slopes when the insertion-fixing portion is forcibly fitted into the coupling space and which are locked to the supporting steps when the insertion-fixing portion is fully inserted in the coupling space, may be formed at the insertion-fixing portion of the head receiver.

The patient-specific artificial temporomandibular joint of the present disclosure is manufactured by 3-D printing on the basis of shape data of the temporal bone or the mandible of a patient, so a good implanting effect is provided and the patient quickly recovers.

Further, worn parts can be partially replaced, there is a little burden of maintenance.

### BRIEF DESCRIPTION OF THE DRAWINGS

The above and other objectives, features and other advantages of the present invention will be more clearly understood from the following detailed description when taken in conjunction with the accompanying drawings, in which:

FIG. 1 is a view showing an application example of a patient-specific artificial temporomandibular joint according to a first embodiment of the present disclosure;

FIG. 2 is a perspective view showing the patient-specific artificial temporomandibular joint according to the first embodiment of the present disclosure;

FIG. 3 is an exploded perspective view of the artificial temporomandibular joint shown in FIG. 2;

FIG. 4 is a cross-sectional view taken along line A-A of FIG. 2;

FIG. 5 is an exploded perspective view showing a modification of the patient-specific artificial temporomandibular joint according to the first embodiment of the present disclosure;

FIG. 6 is a view showing a modification of a temporal bone coupling part that can be applied to the patient-specific artificial temporomandibular joint according to the first embodiment of the present disclosure;

FIG. 7 is a view showing the temporal bone coupling part of the patient-specific artificial temporomandibular joint according to the first embodiment of the present disclosure output by a 3D printer;

FIG. 8 is a perspective view showing a patient-specific artificial temporomandibular joint according to a second embodiment of the present disclosure;

FIG. 9 is an exploded perspective view of the patient-specific artificial temporomandibular joint shown in FIG. 8;

FIG. 10 is a view showing the shape of the bottom of a head receiver shown in FIG. 9; and

FIG. 11 is a view showing the state in which an artificial mandible is coupled to the artificial temporomandibular joint according to the first and second embodiments of the present disclosure.

### DETAILED DESCRIPTION OF THE INVENTION

Hereinafter, one embodiment of the present disclosure is described in detail with reference to accompanying drawings.

A patient-specific artificial temporomandibular joint of the present disclosure is manufactured by 3-D printing on the basis of the shape data of the temporal bone and the mandible of a patient, so a good implanting effect is provided and the patient correspondingly quickly recovers.

The artificial temporomandibular joint of the present disclosure has a fundamental structure composed of: a temporal bone coupling part including a fixing body that is fixed to the temporal bone of a patient and a head receiver that is detachably coupled to the fixing body and provides a close-contact curved surface being open downward; and a mandible coupling part including a coupling body that is fixed to the mandible of a patient and a head that has a spherical shape and is supported by the coupling body in contact with the close-contact curved surface of the head receiver.

The patient-specific artificial temporomandibular joint of the present disclosure may include: a temporal bone coupling part including a fixing body that is fixed to the temporal bone of a patient and a head that has a spherical shape and is detachably coupled to the bottom of the fixing body; and a mandible coupling part including a coupling body that is coupled to the mandible of a patient and a head receiver that is fixed to the coupling body and provides a close-contact curved surface that is in contact with the head.

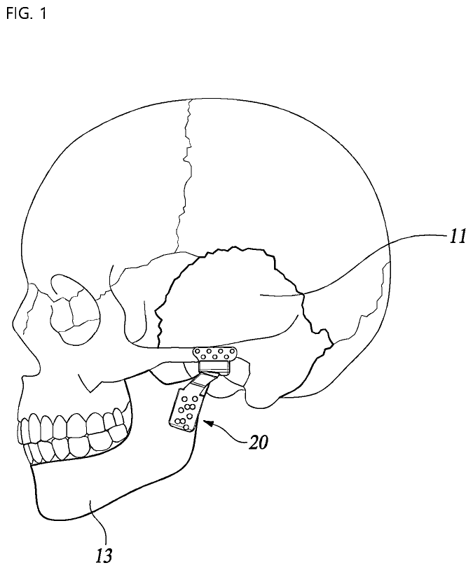

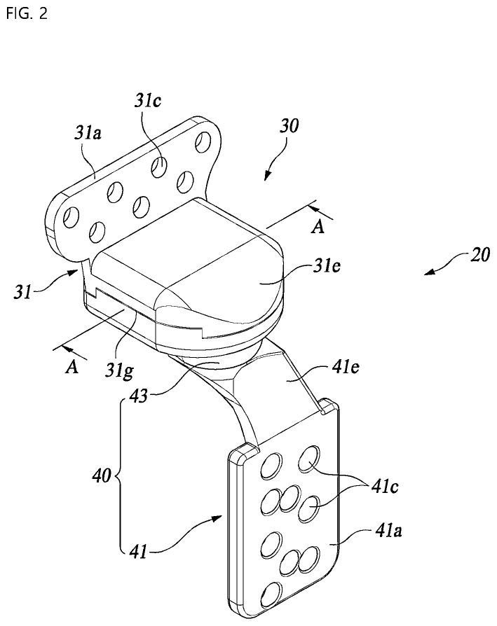

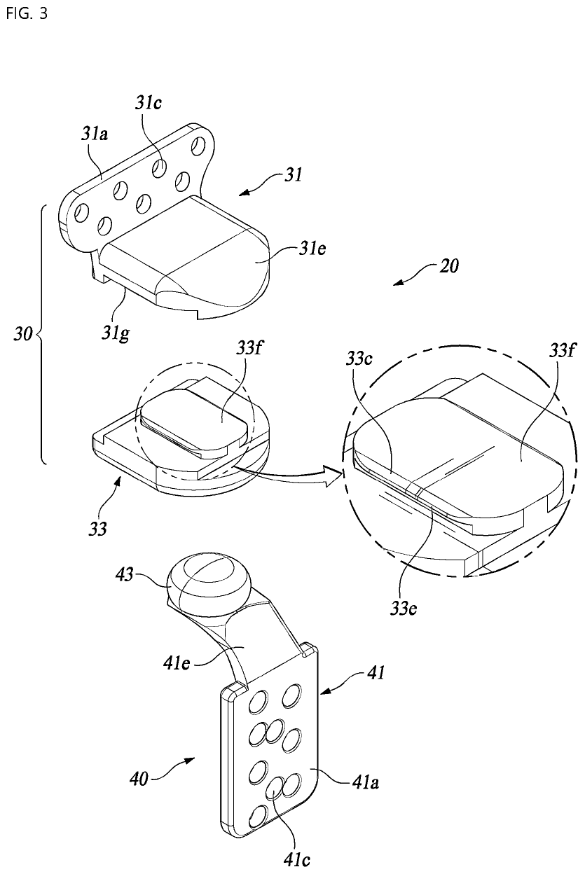

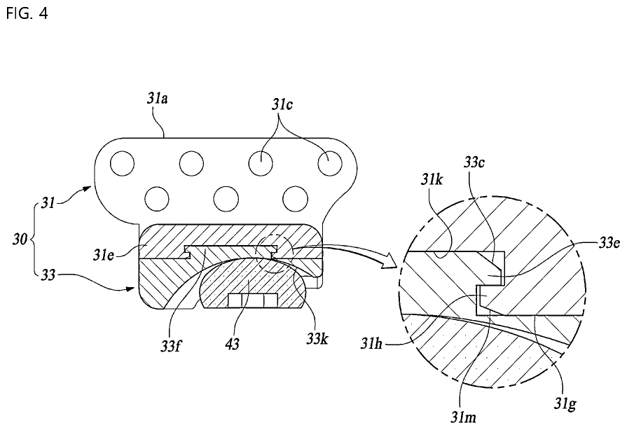

FIG. 1 is a view showing an application example of a patient-specific artificial temporomandibular joint according to a first embodiment of the present disclosure and FIG. 2 is a perspective view showing the patient-specific artificial temporomandibular joint according to the first embodiment of the present disclosure. FIG. 3 is an exploded perspective view of the artificial temporomandibular joint shown in FIG. 2 and FIG. 4 is a cross-sectional view taken along line A-A of FIG. 2.

As shown in the figures, a patient-specific artificial temporomandibular joint 20 according to a first embodiment includes a temporal bone coupling part 30 that is fixed to the temporal bone 11 of a patient, and a mandible coupling part 40 that is coupled to the mandible 13. The temporal bone coupling part 30 and the mandible coupling part perform the function of a temporomandibular joint in contact with each other.

The temporal bone coupling part 30 is formed using a 3D printer and is composed of a fixing body 31 and a head receiver 33. The temporal bone coupling part 30 may be made of cobalt chrome, titanium, stainless steel, or UHMWPE (UltraHigh Molecular Weight Polyethylene).

The fixing body 31 has a fixing plate 31a and a holding supporter 31e. The fixing plate 31a, which is a part that is brought in close contact with the temporal bone of a patient, has several screw holes 31c. The fixing plate 31a is brought in close contact with the temporal bone and then fixed by screws. The fixing body 31 is obtained by 3D printing to correspond to the temporal bone of a patient, so the fixing plate 31a is brought in close contact with the temporal bone without a gap therebetween.

The holding supporter 31e, which is a part coupled to the head receiver, has a coupling space 31k at the center of a close-contact flat surface 31g. The close-contact flat surface 31g is a flat surface being in surface contact with the top of the head receiver 33.

The coupling space 31k, which is a groove for receiving an insertion-fixing portion 33f formed at the head receiver 33, is open downward. The insertion-fixing portion 33f is forcibly fitted in the coupling space 31k, whereby the head receiver 33 is coupled to the holding supporter 31e. The head receiver 33 can be forcibly pulled out of the holding supporter 31e. This will be described below.

Two guide slopes 31m that are parallel with each other are formed around the coupling space 31k. The guide slopes 31m are positioned between the inside of the coupling space 31k and the close-contact flat surface 31g and are inclined toward the coupling space 31k from the close-contact flat surface 31g. The guide slopes 31m guide the insertion-fixing portion 33f into the coupling space when the insertion-fixing portion 33f is forcibly fitted into the coupling space.

Supporting steps 31h are formed inside the coupling space 31k. The supporting steps 31h support elastic locking steps 33e of the insertion-fixing portion fitted in the coupling space 31k.

The head receiver 33, which is a member made of medical synthetic resin, has the insertion-fixing portion 33f on the top and a close-contact curved portion 33k on the bottom. The head receiver may be made of PE-based materials, and particularly, may be made of UHMWPE. The head receiver 33 can elastically deform and can be separated from the fixing body 31 when it is worn. When the head receiver 33 is worn due to long-time use of the artificial temporomandibular joint 20 according to the present disclosure, it is possible to replace only the head receiver 33.

The insertion-fixing portion 33f, which is a portion that is fitted in the coupling space 31k of the holding supporter 31e, has the elastic locking steps 33e and inclined surfaces 33c at both sides.

The inclined surfaces 33c are sliding surfaces that are guided in contact with the guide slopes 31m when the insertion-fixing portion 33f is forcibly fitted into the coupling space 31k. The inclined surfaces 33c slide along the guide slopes 31m when the insertion-fixing portion 33f is forcibly fitted into the coupling space 31k. The elastic locking steps 33e are portions that are elastically deformed by reaction force applied from the guide slopes 31m when the insertion-fixing portion 33f is forcibly fitted into the coupling space and that are elastically restored and locked to the supporting steps 31h when the insertion-fixing portion 33f is fully inserted in the coupling space.

The close-contact curved surface 33k, which is a concave groove being open downward, is in contact with the head 43. The close-contact curved surface 33k and the head 43 are in point contact with each other. The reason that the close-contact curved surface 33k and the head 43 are in point contact with each other is for achieving a better joint motion. In other words, the reason is for enabling the head to roll in all directions.

According to the jaw joint unit of the related art described above, the implant and the second plate are in linear contact with each other, so the implant can be moved only forward and backward. When the jaw is moved left and right, the implant is pulled out of the second plate.

According to this embodiment, since the head 43 can be rotated in any direction in point contact with the close-contact curved surface 33k, the jaw can be moved not only up and down, but left and right, so there is no limit in mastication when a patient eats food.

The mandible coupling part 40 is composed of the coupling body 41 and the head 43. The coupling body 41 and the head 43 are integrated. The mandible coupling part 40 may be made of cobalt chrome, titanium, or stainless steel. Similar to the fixing body 31, the mandible coupling part 40 is also manufactured by 3D printing.

The coupling body 41 has a fixing plate 41a and an extension 41e. The fixing plate 41, which is a part that is fixed to the mandible of a patient by several screws (not shown), has screw holes 41c. Obviously, screws are inserted in the screw holes 41c. The extension 41e is a curved plate integrally formed with the upper end of the fixing plate 41a and extending toward the head receiver 33. The extension 41e functions as a supporter that supports the head 43 under the head receiver 33.

The head 43, which is a member having an elliptical spherical shape, is supported on the coupling body 41 in point contact with the close-contact curved surface 33k of the head receiver 33.

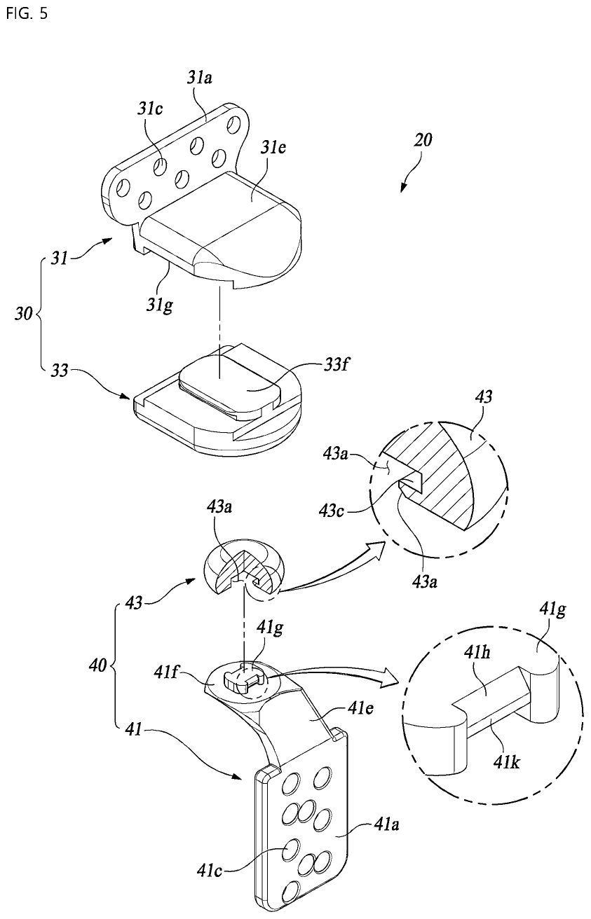

FIG. 5 is an exploded perspective view showing a modification of the patient-specific artificial temporomandibular joint according to the first embodiment of the present disclosure.

In the patient-specific artificial temporomandibular joint 20 shown in FIG. 5, the coupling body 41 and the head 43 can be separated. In this separable type, the coupling body 41 is made of cobalt chrome, titanium, or stainless steel and the head 43 is made of UHMWPE. Since it is a separable type, the head 43 can be separately replaced. The head 43 can elastically deform.

As shown in FIG. 5, a heat support 41f and a fixing protrusion 41g are formed at the upper end of the extension 41e. The head support 41f supports the bottom of the head 43, thereby preventing the head 43 from inclining forward, backward, left, and right.

The fixing protrusion 41g, which is a part protruding upward at the center of the head support 41f, is fitted in a locking groove 43a of the head 43. A slope 41h and a supporting step 41k are formed on each of two sides of the fixing protrusion 41g.

The slopes 41h, which are portions inclined downward and outward from the fixing protrusion 41g, correspond to inclined surfaces 43e of the head 43. The supporting steps 41k lock the elastic locking steps 43c, thereby keeping the head 43 and the fixing protrusion 41g coupled to each other.

The locking groove 43a is formed at the center of the bottom of the head 43. The locking groove 43a, which is a groove in which the fixing protrusion 41g is fitted, has the inclined surfaces 43e and the elastic locking steps 43c. The inclined surfaces 43e, which are portions corresponding to the slopes 41h, slide down on the slopes 41h when the head 43 is pressed to the head support 41f.

The elastic locking steps 43c is inserted under the supporting steps 41k when the head 43 is fully moved down, thereby maintaining the coupling state. The elastic locking steps 43c are fixed by being locked to the supporting steps 41k.

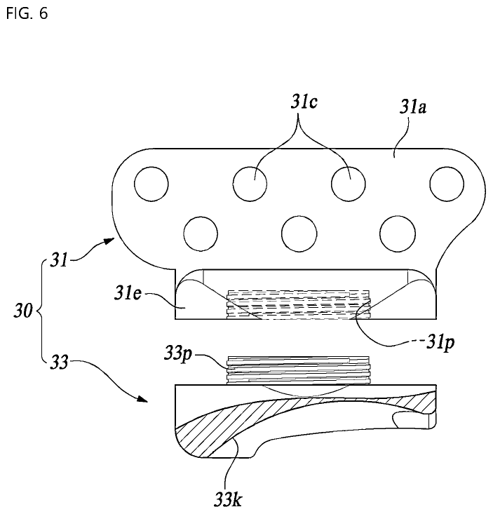

FIG. 6 is a view showing a modification of a temporal bone coupling part that can be applied to the patient-specific temporomandibular joint according to the first embodiment of the present disclosure.

Referring to the figure, it can be seen that a female thread hole 31p is formed in the bottom of the holding supporter 31e and a male thread portion 33p is formed on the top of the head receiver 33. Since the female thread hole 31p and the male thread portion 33p are applied, as described above, the fixing body 31 and the head receiver 33 can be thread-fastened to each other.

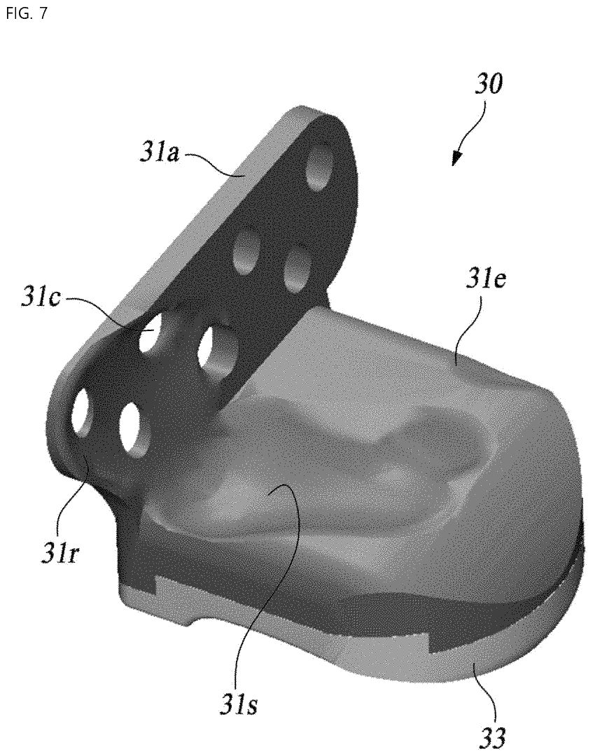

FIG. 7 is a view showing the temporal bone coupler of the patient-specific artificial temporomandibular joint according to the first embodiment of the present disclosure output by a 3D printer.

As show in the figure, grooves 31r and 31s are formed on the fixing plate 31a and the holding supporter 31e, respectively. The grooves 31r and 31s are formed by a 3D printer on the basis of the shape data of the temporal bone and the mandible of a patient. Since an optimal shape suitable for the condition of a patient is precisely formed by 3-D printing, a more efficient surgical operation is possible and satisfaction of a patient can be considerably improved.

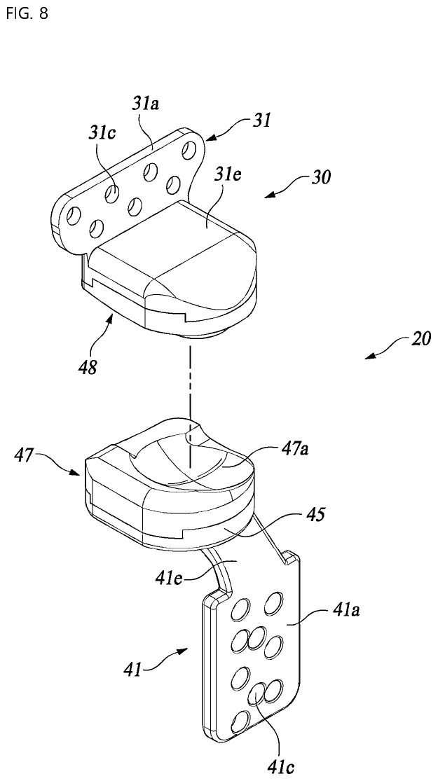

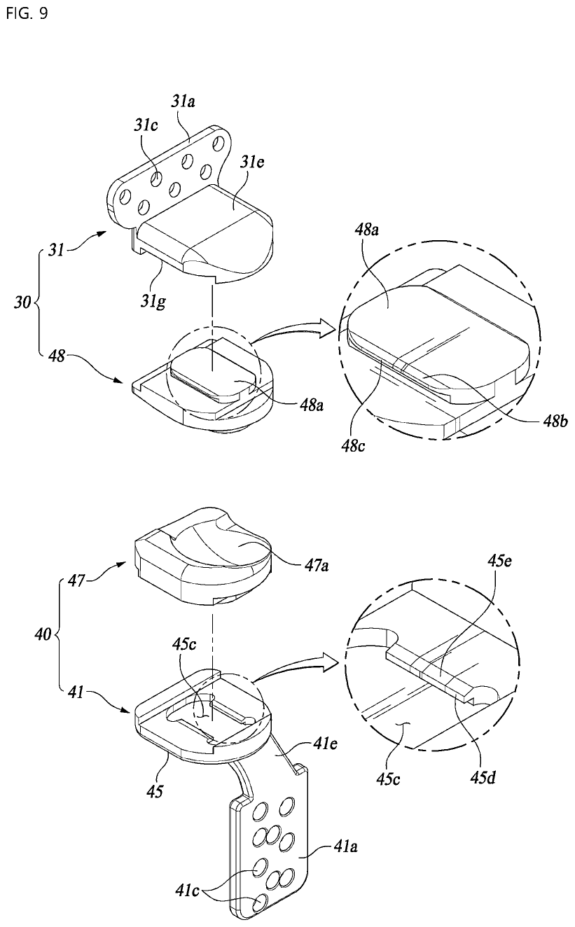

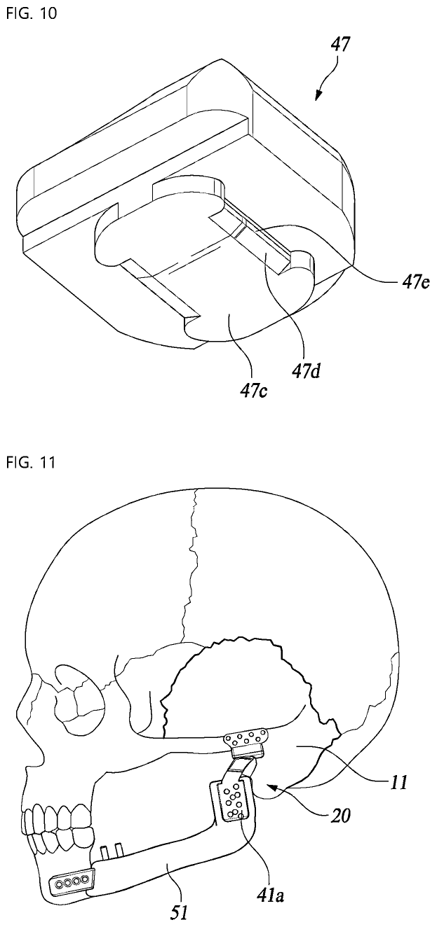

FIG. 8 is a perspective view showing a patient-specific artificial temporomandibular joint 20 according to a second embodiment of the present disclosure and FIG. 9 is an exploded perspective view of the patient-specific artificial temporomandibular joint shown in FIG. 8. FIG. 10 is a view showing the shape of the bottom of a head receiver shown in FIG. 9.

Hereafter, the same members having the same functions are indicated by the same reference numerals as those described above.

As shown in the figures, a patient-specific artificial temporomandibular joint 20 according to a second embodiment of the present disclosure includes a temporal bone coupling part 30 having a head 48 and a mandible coupling part 40 having a head receiver 47. The positions of the head and the head receiver in the artificial temporomandibular joint 20 according to the second embodiment are opposite to those of the first embodiment.

The temporal bone coupling part 30 has a fixing body 31 and the head 48. The fixing body 31 has a fixing plate 31a having screw holes 31c, and a holding supporter 31e. A coupling space is formed on the bottom of the holding supporter 31e. The material or the structure of the fixing body 31 is the same as that described above with reference to FIG. 3.

The head 38, which is detachably coupled to the coupling space of the holding supporter 31e, has an insertion-fixing portion 48a on the top. The insertion-fixing portion 48a is fitted in the coupling space of the holding supporter 31e.

The insertion-fixing portion 48a has the same shape and function as those of the insertion-fixing portion 33f shown in FIG. 3. An inclined surface 48b and an elastic locking step 48c are formed on each of two sides of the insertion-fixing portion 48a. The inclined surfaces 48b correspond to guide slopes 31m at two sides of the coupling space and the elastic locking steps 48c are portion that are fitted on the supporting steps 31h. When the insertion-fixing portion 48a are forcibly fitted into the fixing body 31, the elastic locking steps 48c are inserted into the coupling space 31k and then fixed by being locked to the supporting steps 31h. The head 48 can be separated from the fixing body 31.

The mandible coupling part 40 is composed of the coupling body 41 and a head receiver 47. The coupling body 41 has a fixing plate 41a, an extension 41e, and an adaptor 45.

The adaptor 45 is fixed at the upper end of the extension 41e and has a coupling space 45 being open upward. The coupling space 45c, which is a groove in which the insertion-fixing portion (47c in FIG. 10) formed on the bottom of the head receiver 47 is received and fixed, has a guide slope 45e and a supporting step 45d on each of two sides.

The guide slopes 45e, which correspond to the inclined surfaces 47d of the insertion-fixing portion 47c, slide under the elastic coupling steps 47e when the head receiver 47 is forcibly fitted into the adaptor 45. When the insertion-fixing portion 47c is fully inserted in the coupling space 45c, the supporting steps 45d lock the elastic locking steps 47e, thereby preventing the head receiver 47 from being pulled out.

The head receiver 47, which is a member having a close-contact curved surface 47a on the top and the insertion-fixing portion 47c on the bottom, is detachably coupled to the adaptor 45. The head receiver 47 may be made of UHMWPE.

The close-contact curved surface 47a, which is a groove that is brought in point contact with the head 48, is positioned vertically under the head 48. The insertion-fixing portion 47c, which is a protrusion that is fitted in the coupling space 45c, has an inclined surface 47d and the elastic coupling step 47e on each of two sides. The elastic locking steps 47e are portions that are fitted and fixed under the supporting steps 45d when the insertion-fixing portion 47c is fully inserted in the coupling space 45c.

FIG. 11 is a view showing for reference the state in which an artificial mandible is coupled to the artificial temporomandibular joint according to the first and second embodiments of the present disclosure.

As shown in the figure, the artificial mandible 51 is fixed to the fixing plate 41a of the coupling body 41. Even though the artificial mandible 51 is applied in accordance with the conditions of a patient, the artificial temporomandibular joints of the first and second embodiment can be applied.

Although the present disclosure was described in detail through a detailed embodiment, the present disclosure is not limited thereto and may be modified in various ways by those skilled in the art without departing from the spirit of the present disclosure.

What is claimed is:

1. A patient-specific artificial temporomandibular joint comprising:
a temporal bone coupling part including a fixing body that is fixed to the temporal bone of a patient and a head receiver that is detachably coupled to the fixing body and provides a close-contact curved surface being open downward; and
a mandible coupling part including a coupling body that is fixed to the mandible of a patient and a head that has a spherical shape and is supported by the coupling body in contact with the close-contact curved surface of the head receiver.

2. The patient-specific artificial temporomandibular joint of claim 1, wherein the fixing body is manufactured by a 3D printer or is shaped through machining and has a fixing plate that is brought in contact with the temporal bone and has several screw holes, and a holding supporter that is integrated with the fixing plate and has a coupling space on a bottom thereof, and
the head receiver is made of medical synthetic resin, has an insertion-fixing portion on a top thereof that is detachably mounted in the coupling space, and has the close-contact curved surface on a bottom thereof.

3. The patient-specific artificial temporomandibular joint of claim 2, wherein the coupling body is manufactured by 3D printing or is shaped through machining and has a fixing plate that is fixed to the mandible of a patient and has several screw holes, and an extension that is integrated with the fixing plate and extends under the head receiver.

4. The patient-specific artificial temporomandibular joint of claim 3, wherein the head is made of medical synthetic resin and is detachably coupled to an end of the extension.

5. The patient-specific artificial temporomandibular joint of claim 4, wherein a head support and a fixing protrusion that provide supporting force are formed at the end of the extension, and
a locking groove receiving the fixing protrusion to maintain coupling to the fixing protrusion is formed at the head.

6. The patient-specific artificial temporomandibular joint of claim 2, wherein the coupling space is a groove being open downward to receive the insertion-fixing portion,
guide slopes that guide the insertion-fixing portion into the coupling space when the insertion-fixing portion is forcibly fitted into the coupling space are formed around the coupling space, and
supporting steps that prevent separation of the insertion-fixing portion fitted in the coupling space by supporting the insertion-fixing portion are formed inside the coupling space.

7. The patient-specific artificial temporomandibular joint of claim 6, wherein elastic locking steps, which are elastically deformed by reaction force applied from the guide slopes when the insertion-fixing portion is forcibly fitted into the coupling space and which are locked to the supporting steps when the insertion-fixing portion is fully inserted in the coupling space, are formed at the insertion-fixing portion.

8. The patient-specific artificial temporomandibular joint of claim 2, wherein the coupling space is a female thread hole being open downward and having a predetermined inner diameter and the insertion-fixing portion is a male thread portion that is thread-fastened to the female thread groove.

9. A patient-specific artificial temporomandibular joint comprising:
a temporal bone coupling part including a fixing body that is fixed to the temporal bone of a patient and a head that has a spherical shape and is detachably coupled to a bottom of the fixing body; and
a mandible coupler including a coupling body that is coupled to the mandible of a patient and a head receiver that is fixed to the coupling body and provides a close-contact curved surface that is in contact with the head.

10. The patient-specific artificial temporomandibular joint of claim 9, wherein the fixing body is manufactured by a 3D printer or is shaped through machining and has a fixing plate that is brought in contact with the temporal bone of a patient and has several screw holes, and a holding supporter that is integrated with the fixing plate and provides a coupling space on a bottom thereof, and
the head is made of medical synthetic resin and has an insertion-fixing portion on a top thereof that is detachably coupled to the coupling space.

11. The patient-specific artificial temporomandibular joint of claim 10, wherein the coupling body is manufactured by 3D printing or is shaped through machining and has a fixing plate that is fixed to the mandible of a patient and has several screw holes, and an extension that is integrated with the fixing plate and extends under the head.

12. The patient-specific artificial temporomandibular joint of claim 11, wherein an adaptor having a coupling space being open upward is formed at an extending end of the extension, and the head receiver is made of synthetic resin and has an insertion-fixing portion that is detachably coupled to the coupling space.

13. The patient-specific artificial temporomandibular joint of claim 12, wherein the coupling space is a groove being open to be able to receive the insertion-fixing portion, guide slopes that guide the insertion-fixing portion into the coupling space are formed around the coupling space, supporting steps that support the insertion-fixing portion fitted in the coupling space and prevent separation of the insertion-fixing portion are formed inside the coupling space, and elastic locking steps, which are elastically deformed by reaction force applied from the guide slopes when the insertion-fixing portion is forcibly fitted into the coupling space and which are locked to the supporting steps when the insertion-fixing portion is fully inserted in the coupling space, are formed at the insertion-fixing portion of the head receiver.
